(19) United States
(12) Patent Application Publication
(10) Pub. No.: US 2025/0009949 A1
(43) Pub. Date: Jan. 9, 2025

(54) PERITONEAL DIALYSIS SYSTEM HAVING A CAPILLARY PATIENT LINE FILTER

(71) Applicants: BAXTER INTERNATIONAL INC., Deerfield, IL (US); BAXTER HEALTHCARE SA, Glattpark (Opfikon) (CH)

(72) Inventors: Steffen WAGNER, Deerfield, IL (US); Ralf FLIEG, Deerfield, IL (US); Reinhold BUCK, Deerfield, IL (US); Christof BECK, Deerfield, IL (US); Rainer BLICKLE, Deerfield, IL (US); Bernd KRAUSE, Deerfield, IL (US); Torsten KNOER, Deerfield, IL (US)

(21) Appl. No.: 18/709,360

(22) PCT Filed: Nov. 18, 2022
(86) PCT No.: PCT/US2022/080121

§ 371 Date: May 10, 2024

Related U.S. Application Data
(60) Provisional application No. 63/291,010, filed on Dec. 17, 2021.

(51) Int. Cl. A61M 1/28; B01D 63/02

(52) U.S. Cl. CPC A61M 1/28; B01D 63/02; A61M 2205/0205; A61M 2205/0216; A61M 2205/3327; A61M 2205/3331; A61M 2205/7536; A61M 2206/10; B01D 2221/10; B01D 2313/041; B01D 2313/13

(57) ABSTRACT

A peritoneal dialysis (“PD”) system (10) includes a PD machine (20); a patient line (50) (which may be a dual lumen patient line) extending from the PD machine (20); a filter set (100) in fluid communication with the patient line (50), the filter set (100) including a plurality of hollow fiber membranes (120, e.g., sterilizing grade or bacteria reduction hollow fiber membranes) positioned and arranged such that fresh PD fluid flows through porous walls of the hollow fiber membranes (120) prior to exiting the filter set (100).

### PRIORITY CLAIM

The present application claims priority to and the benefit of U.S. Provisional Application No. 63/291,010, filed on Dec. 17, 2021, the entire contents of which are hereby incorporated by reference.

### BACKGROUND

The present disclosure relates generally to medical fluid treatments and in particular to the filtering of treatment fluid during dialysis fluid treatments.

Due to various causes, a person's renal system can fail. Renal failure produces several physiological derangements. It is no longer possible to balance water and minerals or to excrete daily metabolic load. Toxic end products of metabolism, such as, urea, creatinine, uric acid and others, may accumulate in a patient's blood and tissue.

Reduced kidney function and, above all, kidney failure is treated with dialysis. Dialysis removes waste, toxins and excess water from the body that normal functioning kidneys would otherwise remove. Dialysis treatment for replacement of kidney functions is critical to many people because the treatment is lifesaving.

One type of kidney failure therapy is Hemodialysis (“HD”), which in general uses diffusion to remove waste products from a patient's blood. A diffusive gradient occurs across the semi-permeable dialyzer between the blood and an electrolyte solution called dialysate or dialysis fluid to cause diffusion.

Hemofiltration (“HF”) is an alternative renal replacement therapy that relies on a convective transport of toxins from the patient's blood. HF is accomplished by adding substitution or replacement fluid to the extracorporeal circuit during treatment. The substitution fluid and the fluid accumulated by the patient in between treatments is ultrafiltered over the course of the HF treatment, providing a convective transport mechanism that is particularly beneficial in removing middle and large molecules.

Hemodiafiltration (“HDF”) is a treatment modality that combines convective and diffusive clearances. HDF uses dialysis fluid flowing through a dialyzer, similar to standard hemodialysis, to provide diffusive clearance. In addition, substitution solution is provided directly to the extracorporeal circuit, providing convective clearance.

Most HD, HF, and HDF treatments occur in centers. A trend towards home hemodialysis (“HHD”) exists today in part because HHD can be performed daily, offering therapeutic benefits over in-center hemodialysis treatments, which occur typically bi- or tri-weekly. Studies have shown that more frequent treatments remove more toxins and waste products and render less interdialytic fluid overload than a patient receiving less frequent but perhaps longer treatments. A patient receiving more frequent treatments does not experience as much of a down cycle (swings in fluids and toxins) as does an in-center patient, who has built-up two or three days' worth of toxins prior to a treatment. In certain areas, the closest dialysis center can be many miles from the patient's home, causing door-to-door treatment time to consume a large portion of the day. Treatments in centers close to the patient's home may also consume a large portion of the patient's day. HHD can take place overnight or during the day while the patient relaxes, works or is otherwise productive.

Another type of kidney failure therapy is peritoneal dialysis (“PD”), which infuses a dialysis solution, also called dialysis fluid or PD fluid, into a patient's peritoneal chamber via a catheter. The PD fluid comes into contact with the peritoneal membrane in the patient's peritoneal chamber. Waste, toxins and excess water pass from the patient's bloodstream, through the capillaries in the peritoneal membrane, and into the PD fluid due to diffusion and osmosis, i.e., an osmotic gradient occurs across the membrane. An osmotic agent in the PD fluid provides the osmotic gradient. Used PD fluid is drained from the patient, removing waste, toxins and excess water from the patient. This cycle is repeated, e.g., multiple times.

There are various types of peritoneal dialysis therapies, including continuous ambulatory peritoneal dialysis (“CAPD”), automated peritoneal dialysis (“APD”), tidal flow dialysis and continuous flow peritoneal dialysis (“CFPD”). CAPD is a manual dialysis treatment. Here, the patient manually connects an implanted catheter to a drain to allow used PD fluid to drain from the patient's peritoneal cavity. The patient then switches fluid communication so that the patient catheter communicates with a bag of fresh PD fluid to infuse the fresh PD fluid through the catheter and into the patient. The patient disconnects the catheter from the fresh PD fluid bag and allows the PD fluid to dwell within the patient's peritoneal cavity, wherein the transfer of waste, toxins and excess water takes place. After a dwell period, the patient repeats the manual dialysis procedure, for example, four times per day. Manual peritoneal dialysis requires a significant amount of time and effort from the patient, leaving ample room for improvement.

APD is similar to CAPD in that the dialysis treatment includes drain, fill and dwell cycles. APD machines, however, perform the cycles automatically, typically while the patient sleeps. APD machines free patients from having to manually perform the treatment cycles and from having to transport supplies during the day. APD machines connect fluidly to an implanted catheter, to a source or bag of fresh PD fluid and to a fluid drain. APD machines pump fresh PD fluid from a dialysis fluid source, through the catheter and into the patient's peritoneal chamber. APD machines also allow for the PD fluid to dwell within the chamber and for the transfer of waste, toxins and excess water to take place. The source may include multiple liters of dialysis fluid, including several solution bags.

APD machines pump used PD fluid from the patient's peritoneal cavity, though the catheter, to drain. As with the manual process, several drain, fill and dwell cycles occur during dialysis. A “last fill” may occur at the end of the APD treatment. The last fill fluid may remain in the peritoneal chamber of the patient until the start of the next treatment, or may be manually emptied at some point during the day.

PD fluid needs to be sterile or very near sterile because it is injected into the patient's peritoneal cavity, and is accordingly considered a drug. While bagged PD fluid is typically properly sterilized for treatment, PD fluid made online or PD machines or cyclers that employ disinfection may need additional sterilization.

There is accordingly a need for an effective, low cost way of providing additional sterilization to fresh PD fluid before it is delivered to a patient.

### SUMMARY

The present disclosure provides a peritoneal dialysis (“PD”) system having a PD machine or cycler that pumps fresh PD fluid through a patient line to a patient and removes used PD fluid from the patient via the patient line. The patient line may be reusable or disposable and in either case operates with and fluidly communicates with a filter set. If the patient line is reusable, the reusable patient line is connected to the filter set at the time of treatment. If the patient line is disposable, the filter set is merged into the disposable patient line in one embodiment. In either configuration a distal end of the filter set may be connected to the patient's transfer set, which in turn communicates fluidly with the patient's indwelling catheter.

The PD machine or cycler may include a durable PD fluid pump that pumps PD fluid through the pump itself without using a disposable component, or a disposable type PD fluid pump including a pump actuator that actuates a disposable, fluid-contacting pumping component, such as a peristaltic pump tube or a flexible pumping chamber. The PD machine or cycler also includes a plurality of valves, which may likewise be flow-through and durable without operating with a disposable component, or be disposable type valves having valve actuators that actuate a disposable, fluid-contacting valve component, such as a tube segment or a cassette-based valve seat.

The pumps and valves are under the automatic control of a control unit provided by the machine or cycler. In an embodiment, the valves include a fresh PD fluid valve that the control unit opens to allow the PD fluid pump to pump fresh PD fluid through a fresh PD fluid lumen of a dual lumen patient line to the patient. The valves also include a used PD fluid valve that the control unit opens to allow the PD fluid pump to pump used PD fluid from the patient through a used PD fluid lumen of the dual lumen patient line. It should be appreciated that while a single PD fluid pump may be used, dedicated fresh and used PD fluid pumps may be used alternatively. Also, a single PD fluid pump may include multiple pumping chambers for more continuous PD fluid flow.

The fresh and used PD fluid lumens may again be reusable or disposable. In the instance in which the fresh and used PD fluid lumens are reusable, the lumens terminate with a connector that connects to a lumen-side connector of the filter set, which may be sealed to (e.g., ultrasonically sealed, heat sealed or solvent bonded) or molded with a body of the filter set. The body is in turn sealed to (e.g., ultrasonically sealed, heat sealed or solvent bonded) or molded with a transfer set-side connector that either connects directly to a mating connector of the patient's transfer set or to a mating connector of a short tube placed between the body and the patient's transfer set. The transfer set-side connector may alternatively simply be a port to which the short tube extends over for welding to the port. The body, lumen-side connector, and transfer set-side connector may be referred to herein as a filter housing.

The lumen-side connector and the body form a fresh PD fluid passageway and a used PD fluid passageway. The fresh PD fluid passageway leads to a wall, such as a circular wall providing or defining a plurality of membrane inlet apertures. The membrane inlet apertures may be provided in any desired quantity, e.g., six to twelve, such as eight, and may be formed in a circular pattern spaced apart in equal angular increments. A hollow fiber or capillary membrane is sealed to, e.g., inside of, each inlet aperture. The hollow fiber or capillary membrane is in one embodiment a sterilizing grade or a bacteria reduction hydrophilic membrane, which may be formed with porous walls having a pore size of about 0.2 micron through which the fresh PD fluid flows for further filtration.

Fresh PD fluid flows through the fresh PD fluid passageway and through the insides of each of the multiple hollow fiber or capillary membranes. Providing multiple hollow fiber or capillary membranes enables the membranes and thus the housing of the filter set to be shorter, while providing the necessary filtration needed over multiple patient fills. A shorter housing is better for patient comfort because the patient is typically sleeping near the filter set during treatment. The hollow fiber or capillary membranes are capped at their distal ends, e.g., by crimping, welding, gluing or capping using individual caps or a single, washer-shaped cap, forcing the fresh PD fluid through the pores of the membranes, thereby final filtering the fresh PD fluid.

The final filtered fresh PD fluid flows from the hollow fiber or capillary membranes into a central area of the body located between the membranes, and from there out the transfer set-side connector into the patient's transfer set, either directly or via a short, flexible tube. The hydrophilic nature of the hollow fiber or capillary membranes prevents air from migrating across the membranes once the membranes are fully wetted with fresh PD fluid and thus serve a secondary final stage air removal purpose. If needed however, it is contemplated to provide one or more hydrophobic membrane prior to the hollow fiber or capillary membranes, e.g., along the fresh PD fluid passageway. The one or more hydrophobic membrane allows air to be vented to atmosphere prior to the fresh PD fluid entering the hollow fiber or capillary membranes, which may improve the performance of the membranes in addition to removing air from the filter set.

It is additionally contemplated to place an air diverting net along the fresh PD fluid passageway, e.g., just upstream of the hollow fiber or capillary membranes, which has mesh openings fine enough to divert air once wetted towards the one or more hydrophobic membrane, but wherein the mesh openings are open enough not to significantly block the flow of fresh PD fluid.

Used PD fluid removed through the patient's transfer set enters the housing of the filter set via the transfer set-side connector and flows under negative pressure through central area of the body, the used PD fluid passageway and the used PD fluid lumen, back to the machine or cycler. The machine or cycler pumps the used PD fluid under positive pressure to drain. The used PD fluid does contact the outside of the hollow fiber or capillary membranes but does so in a tangential manner, wherein fibrin, proteins and other particulates within the patient's effluent do not tend to be trapped by or caught on the membranes. The membranes accordingly remain viable over the course of multiple fills of a treatment prior to being discarded with the filter set.

In light of the disclosure set forth herein, and without limiting the disclosure in any way, in a first aspect of the present disclosure, which may be combined with any other aspect, or portion thereof, a peritoneal dialysis (“PD”) system includes a PD machine; a patient line extending from the PD machine; a filter set in fluid communication with the patient line, the filter set including a plurality of hollow fiber membranes positioned and arranged such that fresh PD fluid flows through porous walls of the hollow fiber membranes prior to exiting the filter set.

In a second aspect of the present disclosure, which may be combined with any other aspect, or portion thereof, the patient line is a dual lumen patient line including a fresh PD fluid lumen placed in fluid communication with a fresh PD fluid passageway of the filter set, the dual lumen patient line further including a used PD fluid lumen placed in fluid communication with a used PD fluid passageway of the filter set.

In a third aspect of the present disclosure, which may be combined with any other aspect, or portion thereof, the used PD fluid passageway is in fluid communication with a central area located between the plurality of hollow fiber membranes, the central area receiving used PD fluid from a transfer set-side connector of the filter set.

In a fourth aspect of the present disclosure, which may be combined with any other aspect, or portion thereof, the fresh PD fluid passageway is in fluid communication with a plurality of inlet apertures formed in a wall of the filter set, the inlet apertures forming fresh PD fluid inlets to the plurality of hollow fiber membranes.

In a fifth aspect of the present disclosure, which may be combined with any other aspect, or portion thereof, the fresh PD fluid passageway is formed at least in part via a fresh PD fluid port, wherein the used PD fluid passageway is formed at least in part via a used PD fluid port, and wherein the fresh and used PD fluid ports are part of a lumen-side connector configured to connect to a patient line connector of the dual lumen patient line.

In a sixth aspect of the present disclosure, which may be combined with any other aspect, or portion thereof, the PD system includes a compressible gasket configured to seal around the fresh and used PD fluid ports between the lumen-side connector and the patient line connector.

In a seventh aspect of the present disclosure, which may be combined with any other aspect, or portion thereof, the hollow fiber membranes are closed on one end to force fresh PD fluid through their porous walls, the ends closed individually, or wherein at least two of the ends are closed via a common structure.

In an eighth aspect of the present disclosure, which may be combined with any other aspect, or portion thereof, the PD system includes at least one hydrophobic membrane positioned to vent air from the fresh PD fluid prior to reaching the hollow fiber membranes.

In a ninth aspect of the present disclosure, which may be combined with any other aspect, or portion thereof, the PD system includes at least one net positioned to divert air towards the at least one hydrophobic membrane.

In a tenth aspect of the present disclosure, which may be combined with any other aspect, or portion thereof, the filter set is configured to connect directly to a patient's transfer set, or wherein the filter set includes a flexible tube configured to connect to the patient's transfer set.

In an eleventh aspect of the present disclosure, which may be combined with any other aspect, or portion thereof, the filter set is configured such that used PD fluid flows tangentially along the outsides of the hollow fiber membranes.

In a twelfth aspect of the present disclosure, which may be combined with any other aspect, or portion thereof, the filter set is configured such that fresh PD fluid flows inside to outside through the porous walls of the hollow fiber membranes.

In a thirteenth aspect of the present disclosure, which may be combined with any other aspect, or portion thereof, the PD machine includes a pressure sensor positioned to sense the pressure of fresh PD fluid downstream from the hollow fiber membranes during a patient fill.

In a fourteenth aspect of the present disclosure, which may be combined with any other aspect, or portion thereof, the hollow fiber membranes are sterilizing grade hollow fiber membranes or bacteria reduction hollow fiber membranes.

In a fifteenth aspect of the present disclosure, which may be combined with any other aspect, or portion thereof, a filter set includes a body holding a plurality of hollow fiber membranes positioned and arranged such that fresh PD fluid flows through porous walls of the hollow fiber membranes prior to exiting the body; a lumen-side connector configured to connect to a patient line, the lumen-side connector positioned to introduce fresh PD fluid to the body and to receive used PD fluid from the body; and (i) a transfer set-side connector configured to connect to a patient's transfer set, or (ii) a flexible line configured to connect to the patient's transfer set.

In a sixteenth aspect of the present disclosure, which may be combined with any other aspect, or portion thereof, the body includes a fresh PD fluid passageway in fluid communication with a plurality of inlet apertures formed in a wall of the body, the inlet apertures forming fresh PD fluid inlets to the plurality of hollow fiber membranes.

In a seventeenth aspect of the present disclosure, which may be combined with any other aspect, or portion thereof, a filter set includes a body holding a plurality of capillary membranes positioned and arranged such that fresh PD fluid flows through porous walls of the capillary membranes prior to exiting the body; a lumen-side connector configured to connect to a patient line, the lumen-side connector positioned to introduce fresh PD fluid to the body and to receive used PD fluid from the body; and a transfer set-side connector configured to connect to a patient's transfer set, or wherein the filter set includes a flexible line configured to connect to the patient's transfer set.

In an eighteenth aspect of the present disclosure, which may be combined with any other aspect, or portion thereof, any of the features, functionality and alternatives described in connection with any one or more of FIGS. 1 to 6 may be combined with any of the features, functionality and alternatives described in connection with any other of FIGS. 1 to 6.

In light of the above aspects and the description herein, it is accordingly an advantage of the present disclosure to provide a filter set, which operates with a dual lumen patient line.

It is another advantage of the present disclosure to provide a filter set that filters fresh PD fluid and allows used PD fluid to pass without clogging.

It is a further advantage of the present disclosure to provide a filter set that spaces the filter membranes efficiently to reduce size and aid patient comfort.

Additional features and advantages are described in, and will be apparent from, the following Detailed Description and the Figures. The features and advantages described herein are not all-inclusive and, in particular, many additional features and advantages will be apparent to one of ordinary skill in the art in view of the figures and description. Also, any particular embodiment does not have to have all of the advantages listed herein and it is expressly contemplated to claim individual advantageous embodiments separately. Moreover, it should be noted that the language used in the specification has been selected principally for readability and instructional purposes, and not to limit the scope of the inventive subject matter.

### BRIEF DESCRIPTION OF THE FIGURES

FIG. 1 is a schematic view of one embodiment for peritoneal dialysis system having a hollow fiber or capillary patient line filter set of the present disclosure.

FIG. 2 is a perspective view of one embodiment for a hollow fiber or capillary patient line filter set of the present disclosure.

FIG. 3 is a perspective view of the hollow fiber or capillary patient line filter set of FIG. 2 during a patient fill.

FIG. 4 is an elevation view of a wall located within the hollow fiber or capillary patient line filter set of FIG. 2, the wall forming apertures for introducing fresh PD fluid into a plurality of hollow fiber or capillary membranes.

FIG. 5 is a perspective view of the hollow fiber or capillary patient line filter set of FIG. 2 during a patient drain.

FIG. 6 is a sectioned perspective view illustrating the provision of at least one hydrophobic membrane for venting air and at least one net for diverting air to the at least one hydrophobic membrane.

### DETAILED DESCRIPTION

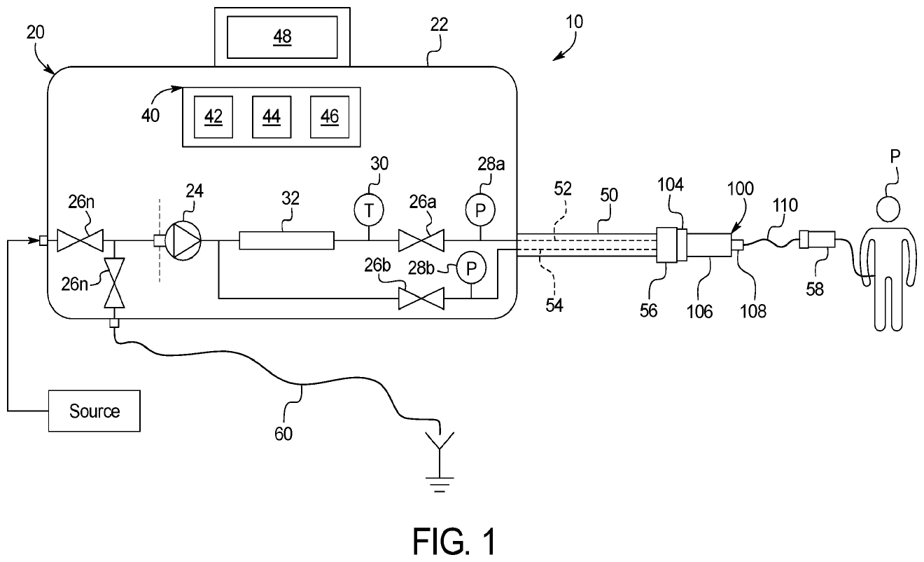

Referring now to the drawings and in particular to FIG. 1, a peritoneal dialysis (“PD”) system 10 is illustrated. PD system 10 includes a PD machine or cycler 20 that pumps fresh PD fluid through a patient line 50 to a patient P and removes used PD fluid from patient P via patient line 50. Patient line 50 may be reusable or disposable and in either case operates with and fluidly communicates with a filter set 100. If patient line 50 is reusable, the reusable patient line is connected to filter set 100 at the time of treatment. If patient line 50 is instead disposable, filter set 100 is merged into or formed with disposable patient line 50 in one embodiment. In either configuration, a distal end of filter set 100 may be connected to the patient's transfer set 58, which in turn communicates fluidly with the indwelling catheter of patient P.

PD machine or cycler 20 may include a housing 22 providing a durable PD fluid pump 24 that pumps PD fluid through the pump itself without using a disposable component. Examples of durable pumps that may be used for PD fluid pump 24 include piston pumps, gear pumps and centrifugal pumps. Certain durable pumps, such as piston pumps are inherently accurate, so that machine or cycler 20 does not require additional volumetric control components. Other durable pumps, such as gear pumps and centrifugal pumps may not be as accurate, such that machine or cycler 20 provides a volumetric control device such as one or more flowmeter (not illustrated).

Pump 24 may alternatively be a disposable type PD fluid pump, which includes a pump actuator that actuates a disposable, fluid-contacting pumping component, such as a peristaltic pump tube or a flexible pumping chamber. Examples of disposable PD fluid pumps that may be used for PD fluid pump 24 include rotary or linear peristaltic pump actuators that actuate tubing, pneumatic pump actuators that actuate cassette sheeting, electromechanical pump actuators that actuate cassette sheeting and platen pump actuators that actuate tubing. It should be appreciated that while a single PD fluid pump 24 may be used, dedicated fresh and used PD fluid pumps may be used alternatively. Also, single PD fluid pump 24 may include multiple pumping chambers for more continuous PD fluid flow.

PD machine or cycler 20 also includes a plurality of valves 26a, 26b, which may likewise be flow-through and durable without operating with a disposable component, or be disposable type valves having valve actuators that actuate a disposable, fluid-contacting valve component, such as a tube segment or a cassette-based valve seat. Examples of durable valves that may be used for valves 26a, 26b include flow-through solenoid valves. Such valves may be two-way or three-way valves. Examples of disposable valves that may be used for valves 26a, 26b include solenoid pinch valves that pinch closed flexible tubing, pneumatic valve actuators that actuate cassette sheeting, and electromechanical valve actuators that actuate cassette sheeting.

Machine or cycler 20 likely includes many valves 26a to 26n. For ease of illustration, machine or cycler 20 is shown having a fresh PD fluid valve 26a that is controlled to open to allow PD fluid pump 24 to pump fresh PD fluid under positive pressure through a fresh PD fluid lumen 52 of dual lumen patient line 50 to patient P. The valves also include a used PD fluid valve 26b that is controlled to open to allow PD fluid pump 24 to pull used PD fluid from patient P under negative pressure through a used PD fluid lumen 54 of dual lumen patient line 50.

Machine or cycler 20 in the illustrated embodiment also includes pressure sensors, such as pressure sensors 28a, 28b. Pressure sensor 28a is located just downstream from fresh PD fluid valve 26a, while pressure sensor 28b is located just upstream from used PD fluid valve 26. Pressure sensor 28a may accordingly sense the pressure in fresh PD fluid lumen 52 of dual lumen patient line 50 even if fresh PD fluid valve 26a is closed, while pressure sensor 28b may sense the pressure in used PD fluid lumen 54 of dual lumen patient line 50 even if used PD fluid valve 26b is closed. Additionally, pressure sensor 28a is positioned to sense the pressure of fresh PD fluid upstream from the filter membranes discussed herein during a patient fill. Pressure sensor 28b perhaps more importantly is positioned to sense the pressure of fresh PD fluid downstream from the filter membranes discussed herein during a patient fill.

Pump 24 and valves 26a, 26b in the illustrated embodiment are under the automatic control of control unit 40 provided by machine or cycler 20 of system 10, while pressure sensors 28a, 28b (and other sensors) output to control unit 40. Control unit 40 in the illustrated embodiment includes one or more processor 42, one or more memory 44 and a video controller 46. Control unit 40 receives, stores and processes signals or outputs from pressure sensors 28a, 28b, and other sensors provided by machine or cycler 20, such as one or more temperature sensor 30 and one or more conductivity sensor (not illustrated). Control unit 40 may use pressure feedback from one or more of pressure sensor 28a, 28b to control PD fluid pump 24 to pump dialysis fluid at a desired pressure or within a safe pressure limit (e.g., within 0.21 bar (three psig) of positive pressure to a patient's peritoneal cavity and −0.10 bar (−1.5 psig) of negative pressure from the patient's peritoneal cavity).

Control unit 40 uses temperature feedback from one or more temperature sensor 30 for example to control a heater 32, such as an inline heater to heat fresh PD fluid to a desired temperature, e.g., body temperature or 37° C. In one embodiment, heater 32 is used additionally to heat a disinfection fluid, such as fresh PD fluid, to disinfect PD fluid pump 24, valves 26a to 26n, heater 32 and all reusable fluid lines within machine or cycler 20 to ready the machine or cycler for a next treatment. The additional filtration discussed herein provides a layer of protection in addition to the heated fluid disinfection to ensure that the PD fluid is safe for delivery to patient P.

Video controller 46 of control unit 40 interfaces with a user interface 48 of machine or cycler 20, which may include a display screen operating with a touchscreen and/or one or more electromechanical button, such as a membrane switch. User interface 48 may also include one or more speaker for outputting alarms, alerts and/or voice guidance commands. User interface 48 may be provided with the machine or cycler 20 as illustrated in FIG. 1 and/or be a remote user interface operating with control unit 40. Control unit 40 may also include a transceiver (not illustrated) and a wired or wireless connection to a network, e.g., the internet, for sending treatment data to and receiving prescription instructions from a doctor's or clinician's server interfacing with a doctor's or clinician's computer.

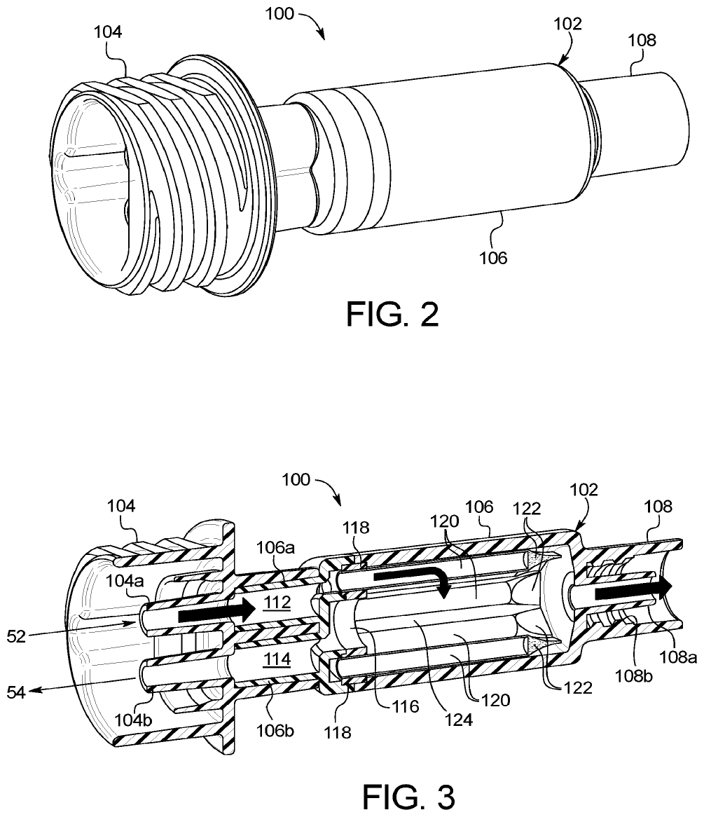

Referring to FIGS. 1 and 2, as mentioned above, fresh and used PD fluid lumens 52 and 54 of dual lumen patient line 50 may again be reusable or disposable. In the instance in which dual lumen patient line 50 is reusable, the lumens terminate with a connector 56 that connects to a lumen-side connector 104 of filter set 100, which may be sealed to (e.g., ultrasonically sealed, heat sealed or solvent bonded) or molded with a body 106 of the filter set. Body 106 is in turn sealed to (e.g., ultrasonically sealed, heat sealed or solvent bonded) or molded with a transfer set-side connector 108 that either connects directly to a mating connector of the patient's transfer set 58 or to a mating connector of a short, flexible tube 110 placed between transfer set-side connector 108 and the patient's transfer set 58.

Referring additionally to FIGS. 3 to 5, transfer set-side connector 108 may include a port 108a and threaded shroud 108b for a luer type connection to a mating connector. Transfer set-side connector 108 may alternatively simply be a port (e.g., port 108a) to which short, flexible tube 110 extends over for welding to the port. Likewise, if dual lumen patient line 50 is disposable, lumen-side connector 104 may alternatively simply include ports, e.g., fresh and used PD fluid ports 104a and 104b, to which fresh and used PD fluid lumens 52 and 54 respectively extend over for welding to the ports. In the illustrated embodiment, ports 104a and 104b are surrounded by a threaded shroud 104c, which may make a luer type connection with mating patient line connector 56. A compressible gasket, e.g., rubber or sponge rubber, not illustrated, may be formed to seal around ports 104a and 104b between patient line connector 56 and lumen-side connector 104. Body 106, lumen-side connector 104 and transfer set-side connector 108 may be referred to herein as a filter housing 102. Filter housing 102 may be made of any one or more plastic, such as, polystyrene (“PS”), polycarbonate (“PC”), blends of polycarbonate and acrylonitrile-butadiene-styrene (“PC/ABS”), polyvinyl chloride (“PVC”), polyethylene (“PE”), polypropylene (“PP”), polyesters like polyethylene terephthalate (“PET”), or polyurethane (“PU”).

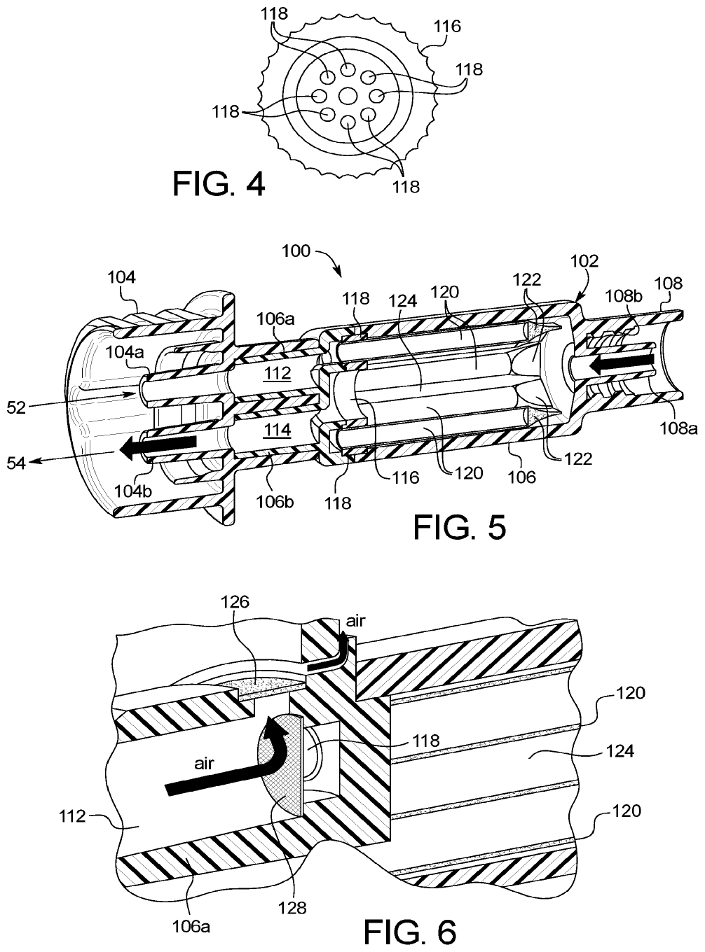

As illustrated in FIG. 3, ports 104a and 104b of lumen-side connector 104 and ports 106a and 106b of body 106 form a fresh PD fluid passageway 112 and a used PD fluid passageway 114, respectively. Fresh PD fluid passageway leads to a wall 116, such as a circular wall, providing or defining a plurality of membrane inlet apertures 118 (FIG. 4). Membrane inlet apertures 118 may be provided in any desired quantity, e.g., six to twelve, such as eight, and may be formed in a circular pattern, wherein the apertures are spaced apart in equal angular increments as illustrated in FIG. 4. A hollow fiber or capillary membrane 120 is sealed to, e.g., inside of, each inlet aperture 118. Each hollow fiber or capillary membrane 120 may be a sterilizing grade or a bacteria reduction grade hydrophilic membrane, which may formed with a porous wall having a pore size of about 0.2 micron through which fresh PD fluid flows for further filtration. Hollow fiber or capillary membranes 120 may be made of, for example, polysulfone or polyethersulfone blended with polyvinylpyrrolidone.

Fresh PD fluid flows through the fresh PD fluid passageway 112 and through the insides of each of the multiple hollow fiber or capillary membranes 120. Providing multiple hollow fiber or capillary membranes 120 enables the membranes and thus housing 102 of filter set 100 to be shorter, while providing the necessary filtration over multiple patient fills. A shorter housing 102 is better for patient comfort because the patient is typically sleeping near filter set 100 during treatment. Hollow fiber or capillary membranes 120 are capped at their distal ends, e.g., by crimping, welding or gluing to form seals 122 as illustrated, or via capping using individual caps or a single, washer-shaped cap (not illustrated), forcing the fresh PD fluid through the pores of membranes 120 as indicated by the curved arrow in FIG. 3, thereby finally filtering the fresh PD fluid.

The final filtered fresh PD fluid flows from hollow fiber or capillary membranes 120 into a central area 124 of body 106 located between membranes 120, and from there out port 108a of transfer set-side connector 108 into the patient's transfer set 58, either directly or via short, flexible tube 110. The hydrophilic nature of hollow fiber or capillary membranes 120 prevents air from migrating across the membranes once the membranes are fully wetted with fresh PD fluid and thus serve a secondary final air removal purpose. As illustrated in FIG. 6, if needed one or more hydrophobic membrane 126, e.g., made from polytetrafluoroethylene (“PTFE”), may be provided prior (from a fresh PD fluid viewpoint) to hollow fiber or capillary membranes 120, e.g., along fresh PD fluid passageway 112. One or more hydrophobic membrane 126 may be welded in place via any of the techniques discussed herein and allows air to be vented to atmosphere prior to the fresh PD fluid entering hollow fiber or capillary membranes 120 as illustrated by the bent air arrows in FIG. 6, which may improve the performance of membranes 120 in addition to removing air from filter set 100.

It is additionally contemplated to fixedly place via any of the welding techniques discussed herein at least one air diverting net 128 along the fresh PD fluid passageway 112, e.g., upstream of the hollow fiber or capillary membranes and downstream from hydrophobic membrane 126 (from a fresh PD fluid viewpoint). Air diverting net 128 has mesh openings fine enough to divert air once the net is wetted towards one or more hydrophobic membrane 126, but wherein the mesh openings of net 128 are open enough not to significantly block the flow of fresh PD fluid. Air diverting net 128 may for example be made of a medically safe metal or a hydrophobic polymer and may have a pore size in the range of from about 0.1 mm to about 0.3 mm.

Used PD fluid removed through the patient's transfer set 58 enters housing 102 of filter set 100 via transfer set-side connector 108 and flows under negative pressure through central area 124 of body 106, used PD fluid passageway 114 and used PD fluid lumen 54, back to machine or cycler 20. Machine or cycler 20 pumps the used PD fluid under positive pressure to drain (e.g., house drain or drain container) via drain line 60. The used PD fluid does contact the outsides of the or capillary membranes 120 but does so in a tangential manner, wherein fibrin, proteins and other particulates within the patient's effluent does not tend to be trapped by or caught on the membranes. Membranes 120 accordingly remain viable over the course of multiple fills of a treatment prior to being discarded with filter set 100.

It should be understood that various changes and modifications to the presently preferred embodiments described herein will be apparent to those skilled in the art. It is therefore intended that any or all of such changes and modifications may be covered by the appended claims. For example, while fresh PD fluid flow is illustrated above as traveling from inside to outside through hollow fiber or capillary membranes 120, it is contemplated for the fresh PD fluid to flow instead from outside to inside through hollow fiber or capillary membranes 120. Here, the finally filtered fresh PD fluid flows through the insides of hollow fiber or capillary membranes 120 into a common collection area within housing 102 prior to exiting through transfer set-side connector 108.

1. A peritoneal dialysis (“PD”) system (10) comprising:
a PD machine (20);
a patient line (50) extending from the PD machine (20);
a filter set (100) in fluid communication with the patient line (50), the filter set (100) including a plurality of hollow fiber membranes (120) positioned and arranged such that fresh PD fluid flows through porous walls of the hollow fiber membranes (120) prior to exiting the filter set (100).

2. The PD system (10) according to claim 1, wherein the patient line (50) is a dual lumen patient line including a fresh PD fluid lumen (52) placed in fluid communication with a fresh PD fluid passageway (112) of the filter set (100), the dual lumen patient line (50) further including a used PD fluid lumen (54) placed in fluid communication with a used PD fluid passageway (114) of the filter set (100).

3. The PD system (10) according to claim 2, wherein the used PD fluid passageway (114) is in fluid communication with a central area (124) located between the plurality of hollow fiber membranes (120), the central area (124) receiving used PD fluid from a transfer set-side connector (108) of the filter set (100).

4. The PD system (10) according to claim 2, wherein the fresh PD fluid passageway (112) is in fluid communication with a plurality of inlet apertures (118) formed in a wall (116) of the filter set (100), the inlet apertures (118) forming fresh PD fluid inlets to the plurality of hollow fiber membranes (120).

5. The PD system (10) according to claim 2, wherein the fresh PD fluid passageway (112) is formed at least in part via a fresh PD fluid port (104a), wherein the used PD fluid passageway (114) is formed at least in part via a used PD fluid port (104b), and wherein the fresh and used PD fluid ports (104a, 104b) are part of a lumen-side connector (104) configured to connect to a patient line connector (56) of the dual lumen patient line (50).

6. The PD system (10) according to claim 5, which includes a compressible gasket configured to seal around the fresh and used PD fluid ports (104a, 104b) between the lumen-side connector (104) and the patient line connector (56).

7. The PD system (10) according to claim 1, wherein the hollow fiber membranes (120) are closed on one end to force fresh PD fluid through their porous walls, the ends closed individually, or wherein at least two of the ends are closed via a common structure.

8. The PD system (10) according to claim 1, which includes at least one hydrophobic membrane (126) positioned to vent air from the fresh PD fluid prior to reaching the hollow fiber membranes (120).

9. The PD system (10) according to claim 8, which includes at least one net (128) positioned to divert air towards the at least one hydrophobic membrane (126).

10. The PD system (10) according to claim 1, wherein the filter set (100) is configured to connect directly to a patient's transfer set, or wherein the filter set (100) includes a flexible tube (110) configured to connect to the patient's transfer set.

11. The PD system (10) according to claim 1, wherein the filter set (100) is configured such that used PD fluid flows tangentially along the outsides of the hollow fiber membranes (120).

12. The PD system (10) according to claim 1, wherein the filter set (100) is configured such that fresh PD fluid flows inside to outside through the porous walls of the hollow fiber membranes (120).

13. The PD system (10) according to claim 1, wherein the PD machine (20) includes a pressure sensor (28b) positioned to sense the pressure of fresh PD fluid downstream from the hollow fiber membranes (120) during a patient fill.

14. The PD system (10) according to claim 1, wherein the plurality of hollow fiber membranes (120) are sterilizing grade hollow fiber membranes or bacteria reduction hollow fiber membranes.

15. A filter set (100) comprising:
a body (106) holding a plurality of hollow fiber membranes (120) positioned and arranged such that fresh PD fluid flows through porous walls of the hollow fiber membranes (120) prior to exiting the body (106);
a lumen-side connector (104) configured to connect to a patient line, the lumen-side connector (104) positioned to introduce fresh PD fluid to the body (106) and to receive used PD fluid from the body (106); and
(i) a transfer set-side connector (108) configured to connect to a patient's transfer set, or (ii) a flexible line (110) configured to connect to the patient's transfer set.

16. The filter set (100) of claim 15, wherein the body (106) includes a fresh PD fluid passageway (112) in fluid communication with a plurality of inlet apertures (118) formed in a wall (116) of the body (106), the inlet apertures (118) forming fresh PD fluid inlets to the plurality of hollow fiber membranes (120).

17. A filter set (100) comprising:
a body (106) holding a plurality of capillary membranes (120) positioned and arranged such that fresh PD fluid flows through porous walls of the capillary membranes (120) prior to exiting the body (106);
a lumen-side connector (104) configured to connect to a patient line, the lumen-side connector (104) positioned to introduce fresh PD fluid to the body (106) and to receive used PD fluid from the body (106); and
a transfer set-side connector (108) configured to connect to a patient's transfer set, or wherein the filter set (100) includes a flexible line (110) configured to connect to the patient's transfer set.
